(19) United States
(12) Patent Application Publication
(10) Pub. No.: US 2023/0203583 A1
(43) Pub. Date: Jun. 29, 2023

(54) BIOMARKERS FOR ASSESSING LIVER DISEASE

(71) Applicant: UNIVERSITY OF FLORIDA RESEARCH FOUNDATION, INCORPORATED, Gainesville, FL (US)

(72) Inventors: NAZLI KHODAYARI, Gainesville, FL (US); MARK LOUIS BRANTLY, Gainesville, FL (US); VIRGINIA CLARK, Gainesville, FL (US)

(21) Appl. No.: 17/923,345

(22) PCT Filed: May 4, 2021
(86) PCT No.: PCT/US2021/030599

§ 371 Date: Nov. 4, 2022

Related U.S. Application Data
(60) Provisional application No. 63/019,971, filed on May 4, 2020.

(51) Int. Cl. C12Q 1/6883; G01N 33/68

(52) U.S. Cl. CPC C12Q 1/6883; G01N 33/6893; C12Q 1/6806

(57) ABSTRACT

Disclosed herein is a method for detecting liver disease in a patient. Also, disclosed are methods of isolating EVs derived from hepatocytes. Methods of assessing effectiveness of liver therapies are also disclosed. Methods involve isolating or otherwise obtaining EVs derived from hepatocytes and analyzing the content of the EVs.

### BACKGROUND

Alpha-1 antitrypsin (AAT), the most abundant serine protease inhibitor in the blood, is primarily produced in the liver (1). AAT is secreted by hepatocytes and contributes to the innate immune system as an anti-inflammatory protein by inhibiting destructive neutrophil proteases, including elastase, cathepsin G, and proteinase 3 (2). Moreover, AAT has been shown to have anti- inflammatory properties independent of its antiprotease activity, affecting many cell types, including neutrophils and macrophages, and has been implicated in modulating cellular processes like apoptosis and cytokine expression (1,3). Alpha-1 antitrypsin deficiency (AATD) is a common inherited cause of liver disease; the most severe mutation form is found in 1 :3500 live births. AATD is the most frequent genetic etiology for pediatric liver disease and transplantation (4). The Z variant of AAT is the most common deficiency allele of the SERPINA1 gene which causes AATD (5). The Z mutation results in accumulation of misfolded ZAAT in hepatocytes and monocytes (6) and low levels of circulating AAT, leading to uncontrolled proteolytic enzyme activity, inducing inflammatory processes in different tissues (7). AATD is mostly associated with the early abundantly secrete ECM proteins, tissue inhibitors of metalloproteinases, and matrix metalloproteinases promoting liver remodeling. In many circumstances, HSC activation is derived from inflammatory mediators released from neighboring cells (16) as well as cells from different organs, such as muscles, intestines, bile ducts, and adipose tissues (14). Although the molecular mechanisms that define fibrogenic processes initiated by HSCs remain incompletely defined, recent studies have explored potential roles for Extracellular Vesicles (EVs) in the pathogenesis of liver fibrosis. Several lines of study have found EVs are important molecular entities involved in cell to cell communication within the liver, contributing to liver pathophysiology (17). EVs are circulating membrane vesicles naturally released from every cell type, with diameters ranging from 30-200 nm. They are enriched in nucleotides, lipids, and proteins from their cells of origin and affect gene expression in recipient cells (18).

Signs of immune-mediated complications, such as panniculitis, cardiovascular risks, and mesangial-capillary glomerulonephritis are uniquely present in AATD patients (5,19,20) and suggest a contribution from a variety of activated immune cells as well as parenchymal cells. Recent research has implicated that EVs released by activated immune cells and injured parenchymal cells act in a paracrine manner to stimulate liver fibrosis (21). This study was conducted to test the hypothesis that dysregulated immune response, as well as activated parenchyma associated with AATD, results in release of EVs carrying pro-fibrotic cargo with the ability to promote liver fibrosis through activation of HSCs. To investigate this, we isolated EVs from plasma of healthy controls and AATD individuals, characterized them, and profiled their cytokine and miRNA expression. We show that the AATD individuals exhibit a pro-fibrogenic EV cytokine and miRNA profile. Furthermore, we show that AATD plasma derived EVs induced HSC transdifferentiation and activation in vitro. Our results suggest that in the pathogenesis of AATD- mediated liver disease, EVs play an important role in modulating a cross-talk network in liver fibrosis.

### BRIEF DESCRIPTION OF THE DRAWINGS

FIG. 1. Isolation and characterization of plasma EVs. FIG. 1 (A) Ultracentrifugation was used to isolate EVs from the plasma of normal (MM) and AATD (ZZ) individuals; FIG. 1 (B) NTA was performed on plasma EVs purified by ultracentrifugation to determine their concentration (upper panel) or size (nm) (lower panel) in individual groups of MM and ZZ (26 individuals per group); FIG. 1 (C) EVs were characterized by TEM; FIG. 1 (D) Western blot analysis of EVs using anti-CD 63 (left), anti-CD81 (right), and anti-TSG 101 antibodies (middle). Graphs show quantification of immunoreactive signals by scanning, normalized to the number of the EVs, *P < 0.05.

FIG. 2. Comparison of EV-associated and plasma cytokines and chemokines between control and AATD subjects. FIG. 2(A) Representative liver biopsies show increasing PAS + D globule grade, indicating ZAAT accumulation. Black arrows highlight cells with PAS-D globules. Scale bar represents 100 µm. FIG. 2(B) The distribution of CRP levels in MM and ZZ individuals. FIG. 2(C) The levels of differential expressed free and EV-associated cytokines in plasma samples from MM individuals, compared to ZZ individuals, n=26, *P < 0.05. FIG. 2(D) The levels of total and polymeric AAT in plasma derived EVs pooled from 4 MM and 4 ZZ individuals. Calnexin (CNX) has been loaded as negative marker for EVs.

FIG. 3. Differential miRNA Expression Profiles among plasma derived EV miRNAs from healthy and AATD individuals. FIG. 3(A) Volcano plot of total and FIG. 3(B) differentially expressed miRNAs in ZZ individuals compared with MM healthy controls. Plotted along the x-axis is the mean of log2 fold-change and the y-axis is the negative logarithm of the -log P-values. Red points represent significantly upregulated miRNAs and blue points represent significantly downregulated miRNAs. FIG. 3(C) Heatmap depicting the expression analysis of all miRNAs detected in EVs from ZZ and healthy MM populations. Green and gray represents miRNAs with lower and higher expression, respectively, after robust multiarray average normalization. FIG. 3(D) Molecular networks linking highly differentially expressed microRNAs (miRNAs) and their target genes involved in hepatic stellate cell activation between AATD individuals and healthy controls. Upregulated miRNAs are in red and downregulated miRNAs are green.

FIG. 4. Plasma derived-EVs from AATD individuals activate hepatic stellate cells in vitro. FIG. 4(A) Fluorescent microscopic images demonstrating interaction of DiO-labeled EVs with recipient HSCs. Images were taken 1 hour after EVs were added to the culture medium. Images were captured 20× objective. Flow cytometric analysis confirmed a population of green fluorescence positive cells present in EV-fed groups. FIG. 4(B) Representative phase contrast images of morphology of quiescent stellate cells incubated with PBS (top row), c (middle row) and ZZ plasma derived- EVs (bottom row) during 24 hours of incubation. FIG. 4(C) LX-2 cells were incubated with and without isolated MM and ZZ plasma derived-EVs and the expression of α-SMA and Col1 a1 were measured using real-time PCR after 24 hours of incubation. FIG. 4(D) Immunofluorescence images of the expression levels of α-SMA in LX-2 cells incubated with and without isolated MM and ZZ plasma derived-EVs. Green fluorescence indicates LX-2 cells that express α-SMA and blue spots indicate nuclei stained with DAPI (Images were captured 40× objective).

FIG. 5. NF-κB and JAK/STAT-dependent regulation of activation and migration of HSCs. FIG. 5A and FIG. 5B. The iPSCs derived-HSCs were incubated with and without isolated MM and ZZ plasma derived-EVs and the protein expression of the phospho-IKK, total IKK, nuclear p65, nuclear p50, cytoplasmic STAT1 and JNK, and nuclear phospho-STAT-1 and phosphor-JNK were determined during 24 hours of incubation by Western blot analysis. GAPDH protein level was used as loading control. The assays were performed at least three times with similar results. FIG. 5(C) Wound-healing and FIG. 5(D) cell proliferation assays of HSCs treated with or without EVs derived from MM and ZZ plasma samples or PBS control during 24 hours of incubation. (E and F) LX-2 cells were incubated with and without isolated MM and ZZ plasma derived-EVs and the expression of CXCL10 and CXCR3 were measured using real-time PCR after 24 hours of incubation.

FIG. 6. High CXCL10 plasma levels is associated with AATD mediated-liver fibrosis in AATD individuals. FIG. 6(A) Dot plot graph illustrating differences in the serum CXCL10 levels of ZZ individuals compared to MM healthy controls, *P < 0.05. FIG. 6(B) The correlation between serum CXCL10 levels and α-SMA immunoreactivity on the liver biopsy from ZZ individuals. FIG. 6(C) Mild and severe perisinusoidal α-SMA staining on the liver biopsy from ZZ individuals with low and high CXCL10 plasma levels respectively.

FIG. 7. Plasma derived EV mediated liver fibrosis in AATD individuals. EVs are small vesicular structures that are shed by different cells and provide various autocrine and paracrine signaling cues. AATD mediated immune complications, such as lung disease, panniculitis, cardiovascular risks, and mesangial-capillary glomerulonephritis suggest a contribution from a variety of activated immune cells as well as parenchymal cells. EVs released by activated immune cells and injured parenchymal cells carry a pro-fibrotic cargo with the ability to promote liver fibrosis through activation of HSCs. AATD plasma derived EVs induced HSC trans differentiation and activation leading to deposition of ECM and fibrotic phenotype.

FIG. 8. Using violet laser equipped CytoFlex flowcytometry allows us to identify exosomes labeled with ASGR1 which is a liver derived marker on the surface of exosomes. Our data shows 30- 40% of plasma derived exosomes are originated from liver in healthy controls. Liver derived exosomes are about 60% of the total plasma derived exosomes. These data also indicates that patients with more liver AAT accumulation have more liver derived exosomes compare to patient 105 which have very low levels of liver AAT accumulation.

### DETAILED DESCRIPTION

### Definitions

“Biomarker” means a biomolecule (e.g. cytokine, factor, miRNA or other nucleic acids, phospholipid) or blood component (e.g. circulating EVs) that is differentially present (i.e., increased or decreased) in a biological sample from a subject or a group of subjects having a first phenotype (liver disease) as compared to a biological sample from a subject or group of subjects having a second phenotype (not having liver disease). A biomarker may be a biomolecule associated with EVs (i.e., contained within or bound to EVs) or in free form (i.e. not contained within or bound to EVs). Thus, in one instance, a biomarker may be differentially present in blood or plasma in free form or in another instance differentially present in EVs. Also, a biomarker may be the EVs themselves that are associated with one or more biomolecules, wherein such EVs are differentially present in a biological sample such as blood, serum, or plasma.

A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch’s T-test or Wilcoxon’s rank-sum Test).

The term “biomarker-associated EVs” as used herein refers to EVs that are bound to or contain a biomolecule biomarker.

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological fluid such as, for example, blood, blood plasma, blood serum, urine, or cerebral spinal fluid (CSF), tissue or tissue homogenate. Another example of a biological includes EVs obtained from or present in blood serum, or plasma.

“Subject” means any animal, but is preferably a mammal, such as, for example, a human, monkey, non-human primate, mouse, or rabbit.

A “reference level” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Steatosis” refers to fatty liver disease without the presence of inflammation. The condition can occur with the use of alcohol or in the absence of alcohol use.

“Non-alcoholic fatty liver disease” (NAFLD) refers to fatty liver disease (steatosis) that occurs in subjects even in the absence of consumption of alcohol in amounts considered harmful to the liver.

“Steatohepatitis” refers to fatty liver disease that is associated with inflammation. Steatohepatitis can progress to cirrhosis and can be associated with hepatocellular carcinoma. The condition can occur with the use of alcohol or in the absence of alcohol use.

“Non-alcoholic steatohepatitis” (NASH) refers to steatohepatitis that occurs in subjects even in the absence of consumption of alcohol in amounts considered harmful to the liver. NASH can progress to cirrhosis and can be associated with hepatocellular carcinoma.

“Fibrosis” refers to the accumulation of extracellular matrix proteins in the liver as a result of ongoing inflammation. Fibrosis is classified histologically in a liver biopsy sample into five stages, 0-4. Stage 0 means no fibrosis, Stage 1 refers to mild fibrosis, Stage 2 refers to moderate fibrosis, Stage 3 refers to severe fibrosis, and Stage 4 refers to cirrhosis.

“Liver disease” or “LD”, as used herein refers to NAFLD, NASH, fibrosis, and/or cirrhosis.

“Liver disease therapy” or “LDT” refers to a therapy directed at treating liver disease. Examples of LDT include administration of one or more agents known to improve or prevent progression of liver disease, including but not limited to, ACE inhibitors, alpha-tocopherol, interferon-alpha, PPAR-alpha agonists, anti-TGF-β1 monoclonal antibody (e.g. Fresolimumab), LOXL2 monoclonal antibody (e.g. AB0023 or GS-6624), IL-4/IL-13 dual antibody, LPA1 receptor antagonists, Av-beta-6 antibody, tyrosine kinase inhibitors, angiotensin receptor blockers, perferidone, obeticholic acid (OCALIVA), TIMP-1 antibody, PPAR-gamma agonists, TGf-beta inhibitors, Human pentraxin-2, Prolyl hydroxylase inhibitors, hedgehog inhibitors, CB1 inhibitors, Caspase inhibitors, or Galactin-3 inhibitor.

“NAFLD Activity Score” or “NAS” refers to a histological scoring system for NAFLD. The score is comprised of evaluation of changes in histological features such as steatosis, lobular inflammation, absence of lipogranulomas, and hepatocyte ballooning. Fibrosis is assessed independently of the NAS.

“Severity” of liver disease refers to the degree of liver disease on the spectrum of non-alcoholic liver disease activity, ranging from low severity disease associated with fat accumulation in the liver (NAFLD), with an increased severity associated with low levels of inflammation and/or fibrosis in addition to fat accumulation (i.e., borderline NASH), and a further increase in severity associated with higher levels of inflammation and fibrosis (i.e., NASH). Severity may be based on fibrosis stages or may also be assessed using the NAS.

Where the definition of terms departs from the commonly used meaning of the term, applicant intends to utilize the definitions provided below, unless specifically indicated.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” is meant to denote up to a 5, 6, 7, 8, 9, or 10 percent variance in the stated value or range. For example, about 2 includes values of 1.9 to 2.1.

As used herein, “extracellular vesicles” encompasses “exosomes,” or “microvesicles (MVs),” which are released by almost all types of cells upon fusion of its multi-vesicular body with a plasma membrane of the cell, in some embodiments. The term “extracellular vesicles” may include both exosomes and MVs. Extracellular vesicles are present in many, if not all, eukaryotic fluids, including blood, urine and cultured medium of cell cultures. Extracellular vesicles, in particular exosomes or MVs, are known for their role in cell to cell communications and have demonstrated an ability to unload their contents and contribute to the transformation of normal and stem cells to cancerous states. Microvesicles, for example, can be formed by a variety of processes, including the release of apoptotic bodies, the budding of microvesicles directly from the cytoplasmic membrane of a cell, and exocytosis from multivesicular bodies. For example, extracellular vesicles are commonly formed by their secretion from the endosomal membrane compartments of cells as a consequence of the fusion of multivesicular bodies with the plasma membrane. The multivesicular bodies (MVBs) are formed by inward budding from the endosomal membrane and subsequent pinching off of small vesicles into the luminal space. The internal vesicles present in the MVBs are then released into the extracellular fluid as so-called exosomes or extracellular vesicles. As part of the formation and release of extracellular vesicles, unwanted molecules are eliminated from cells. However, cytosolic and plasma membrane proteins are also incorporated during these processes into the extracellular vesicles, resulting in extracellular vesicles having particle size properties, lipid bilayer functional properties, and other unique functional properties that allow the vesicles to potentially function to carry their payload.

The terms “treat”, “treating” or “treatment of” as used herein refers to providing any type of medical management to a subject. Treating includes, but is not limited to, administering a composition to a subject using any known method for purposes such as curing, reversing, alleviating, reducing the severity of, inhibiting the progression of, or reducing the likelihood of a disease, disorder, or condition or one or more symptoms or manifestations of a disease, disorder or condition.

As used herein, the term “biologically effective amount” or “therapeutically effective amount” of therapeutic agent is intended to mean a nontoxic but sufficient amount of such therapeutic agents to provide the desired therapeutic effect. The amount that is effective will vary from subject to subject, depending on the age and general condition of the individual, the particular active agent or agents, and the like. Thus, it is not always possible to specify an exact effective amount. However, an appropriate effective amount in any individual case may be determined by one of ordinary skill in the art using routine experimentation.

As used herein, the term “pharmaceutical composition” contemplates compositions comprising one or more therapeutic agents as described above, and one or more pharmaceutically acceptable excipients, carriers, or vehicles. As used herein, the term “pharmaceutically acceptable excipients, carriers, or vehicles” comprises any acceptable materials, and/or any one or more additives known in the art. As used herein, the term “excipients,” “carriers” or “vehicle” refer to materials suitable for drug administration through various conventional administration routes known in the art. Excipients, carriers, and vehicles useful herein include any such materials known in the art, which are nontoxic and do not interact with other components of the composition in a deleterious manner.

The terms “normal” and “healthy” are used herein interchangeably. They refer to an individual or group of control individuals who have not shown any symptoms of NAFLD, NASH, or other liver damage or diseases, such as liver inflammation, fibrosis, steatosis, and have not been diagnosed with NAFLD, NASH, or other liver damage or diseases. The normal individual (or group of individuals) is not on medication affecting NAFLD, NASH, or other liver damage or diseases. In certain embodiments, normal individuals have similar sex, age, body mass index as compared with the individual from which the sample to be tested was obtained. The term “normal” is also used herein to qualify a sample isolated from a healthy individual.

### Exemplary Embodiments

Embodiments described herein provide a non-invasive or minimally invasive method of detecting, monitoring, or assessing the degree, severity, or progression of liver damage in a subject with liver disease. In contrast to prior art diagnostic methods, the disclosed methods are able to readily diagnose liver damage using a bodily sample that is obtained from the subject by non-invasive or minimally invasive methods. The bodily sample can include, for example, bodily fluids, such as blood, serum, or plasma that are obtained by minimally invasive methods. Embodiments can, for example, can be used as a test to distinguish steatosis from non-alcoholic steatohepatitis (NASH), and detect early stages of liver fibrosis.

According to other embodiments, a method is disclosed for monitoring the response of a subject to treatment of liver disease or liver damage and to a method of monitoring the pathogenesis of liver damage caused by an agent administered to a subject. The invention may also be used to detect or monitor the progression of other forms of liver disease.

One aspect of the invention therefore relates to a method of predicting, detecting, monitoring, or assessing the degree or severity of liver disease or other liver damage or diseases in a subject. In certain embodiments, the method includes obtaining a bodily sample from the subject, and determining an amount of one or more biomarkers associated with circulating EVs in the sample. A differentially present level of the one or more biomarkers associated with circulating EVs in the subject compared to a control is indicative of an increase in degree or severity of liver fibrosis, NAFLD and potentially nonalcoholic steatohepatitis (NASH), angiogenesis or other damage in the subject. In certain embodiments, the majority of the circulating EVs are hepatocyte-derived. In these embodiments, a differentially present level of the EVs associated with biomolecules indicative of liver disease or differentially present levels of biomolecules indicative of liver disease in EVs from the subject compared to a control is indicative of an increase in degree or severity of liver disease (e.g. liver fibrosis) or other damage in the subject.

In other embodiments, provided are methods that involve detecting and determining an expression level of at least one biomarker expressed or detected in the circulating EVs in a bodily sample. These biomarkers are involved in molecule function and cellular localization.

In certain embodiments, the bodily sample can comprise a blood sample obtained non-invasively from the subject. In some aspects, the amount of blood taken from a subject is about 0.1 ml or more. In an exemplary embodiment, the bodily sample is blood plasma isolated from a whole blood sample obtained from a subject. Blood plasma may be isolated from whole blood using well known methods, such as centrifugation. The bodily samples can be obtained from the subject using sampling devices, such as syringes, swabs or other sampling devices used to obtain liquid and/or solid bodily samples either invasively (i.e., directly from the subject) or non-invasively. These samples can then be stored in storage containers. The storage containers used to contain the collected sample can comprise a non- surface reactive material, such as polypropylene. The storage containers should generally not be made from untreated glass or other sample reactive material to prevent the sample from becoming absorbed or adsorbed by surfaces of the glass container.

Collected samples stored in the container may be stored under refrigeration temperature. For longer storage times, the collected sample can be frozen to retard decomposition and facilitate storage. For example, samples obtained from the subject can be stored in a falcon tube and cooled to a temperature of about -80° C. The collected bodily sample can be stored in the presence of a chelating agent, such as ethylenediaminetetraacetic acid (EDTA). The collected bodily sample can also be stored in the presence of an antioxidant, such as butylated hydroxytoluene (BHT) or diethylenetriamine pentaacetic acid, and/or kept in an inert atmosphere (e.g., overlaid with argon) to inhibit oxidation of the sample.

Bodily samples obtained from the subject can then be contacted with a solvent, such as an organic solvent. The solvent can include any chemical useful for the removal (i.e., extraction) of the EVs of interest from a bodily sample. For example, where the bodily sample comprises plasma, the solvent can include a water/methanol mixture. It will be appreciated by one skilled in the art that the solvent is not strictly limited to this context, as the solvent may be used for the removal of lipids from a liquid mixture, with which the liquid is immiscible in the solvent. Those skilled in the art will further understand and appreciate other appropriate solvents that can be employed to extract lipids from the bodily sample. The solvent can include solvent mixtures comprising miscible, partially miscible, and/or immiscible solvents. The solvent can also be combined with other solvents which can act as carriers facilitating mixing of the solvent with the bodily sample or transfer of the extracted EVs from the bodily sample.

The bodily sample may be pre-treated as necessary by dilution in an appropriate buffer solution, heparinized, concentrated if desired, or fractionated by any number of methods including, but not limited to, ultracentrifugation, fractionation by fast performance liquid chromatography, or other known methods. Any of a number of standard aqueous buffer solutions, employing one of a variety of buffers, such as phosphate, Tris, or the like at a physiological pH can be used.

After obtaining the bodily sample (e.g., blood, serum, plasma), the amount of biomarker-associated EVs in the bodily sample, or an expression level of one or more biomarkers associated with the EVs, is detected, measured, and/or quantifying to determine the level of biomarker-associated EVs or the biomarkers of interest in the subject. A differential presence of biomarker-associated EVs, or the biomarker of interest is associated with an increase in liver damage and/or liver disease.

In certain embodiments, the circulating biomarker-associated EVs in the bodily sample, as well as biomolecule biomarkers associated therewith, can be detected and/or quantified using an immunoassay, such as an enzyme- linked immunoabsorbent assay (ELISA), or other assays, now known or later developed, that can be used to detect and/or quantify EVs and biomarkers of interest in the bodily sample. These assays include, but are not limited to, flow cytometry (FACS) analysis, radioimmunoassays, both solid and liquid phase, fluorescence- linked assays, competitive immunoassays, mass spectrometry (MS)- based methods (e.g., liquid chromatography MS), and HPLC.

Once the level or amount of the biomarker-associated EVs or the expression level of the biomarker of interest in a sample are determined, the level/amount can be compared to a predetermined value or control value to provide information for diagnosing, monitoring, or assessing liver fibrosis, NAFLD, and/or NASH, in a subject. For example, the level/amount of EVs or the expression level of the biomarker associated therewith in a sample can be compared to a predetermined value or control value to determine if a subject is afflicted with liver fibrosis, NAFLD, NASH, or other liver damage or diseases.

The level/amount of biomarker-associated EVs or the expression level of the biomarkers associated therewith, in the subject’s bodily sample may also be compared to the level/amount of the EVs or the expression level of the biomarkers of interest obtained from a bodily sample previously obtained from the subject, such as prior to administration of therapeutic. Accordingly, the method described herein can be used to measure the efficacy of a therapeutic regimen for the treatment of liver fibrosis, NAFLD, NASH, or other liver damage or diseases in a subject by comparing the level/amount of EVs or the expression level of the biomarkers of interest in bodily samples obtained before and after a therapeutic regimen. Additionally, the methods described herein can be used to measure the progression of liver fibrosis, NAFLD, NASH, or other liver damage or diseases in a subject by comparing the level/amount of EVs or the expression level of the biomarker of interest in a bodily sample obtained over a given time period, such as days, weeks, months, or years.

The level/amount of EVs or the expression level of the biomarker of interest in a sample may also be compared to a predetermined value or control value to provide information for determining the severity of the disease in the subject or the tissue of the subject (e.g., liver tissue). Thus, in some aspect, a level/amount of biomarker-associated EVs or the expression level of the biomarker of interest may be compared to control values obtained from subjects with well-known clinical categorizations, or stages, of histopathologies related to liver fibrosis, NAFLD and/or NASH (e.g., lobular liver inflammation, liver steatosis, and liver fibrosis). In one particular embodiment, a level/amount of biomarker-associated EVs or the expression level of the biomarker of interest in a sample can provide information for determining a particular stage of fibrosis in the subject. For example, stages of fibrosis may be defined as Stage 1: no fibrosis or mild fibrosis; Stage 2: moderate fibrosis; Stage 3 and 4: severe fibrosis.

A predetermined value or control value can be based upon the level/amount of EVs or the expression level of the biomarker of interest in comparable samples obtained from a healthy or normal subject or the general population or from a select population of control subjects. In some aspects, the select population of control subjects can include individuals diagnosed with liver fibrosis, NAFLD and/or NASH. For example, a subject having a greater level/amount of EVs or the expression level of the biomarker of interest compared to a control value may be indicative of the subject having a more advanced stage of a liver histopathology.

The predetermined value can be related to the value used to characterize the level/amount of biomarker-associated EVs or the expression level of the biomarker of interest in the bodily sample obtained from the test subject. Thus, if the level/amount of EVs or the expression level of the biomarker of interest is an absolute value, the predetermined value can also be based upon the absolute value in subjects in the general population or a select population of human subjects. Similarly, if the level/amount of EVs or the expression level of the biomarker of interest is a representative value such as an arbitrary unit, the predetermined value can also be based on the representative value.

The predetermined value can take a variety of forms. The predetermined value can be a single cut-off value, such as a median or mean. The predetermined value can be established based upon comparative groups such as where the level/amount of biomarker-associated EVs or the expression level of the biomarker of interest in one defined group is double the level/amount of EVs or the expression level of the biomarker of interest in another defined group. The predetermined value can be a range, for example, where the general subject population is divided equally (or unequally) into groups, or into quadrants, the lowest quadrant being subjects with the lowest level/amount of EVs or the expression level of the biomarker of interest, the highest quadrant being individuals with the highest level/amount of EVs or the expression level of the biomarker of interest. In an exemplary embodiment, two cutoff values are selected to minimize the rate of false positive and negative results.

Predetermined values of the biomarker-associated EVs or the expression level of the biomarkers associated therewith, such as for example, mean levels, median levels, or “cutoff” levels, are established by assaying a large sample of subjects in the general population or the select population and using a statistical model such as the predictive value method for selecting a positively criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff value can be determined for EVs or the expression level of each biomarker that is assayed.

In other embodiments, the invention relates to a method for generating a result useful in diagnosing and monitoring liver fibrosis, NAFLD, NASH, or other liver damage or diseases by obtaining a dataset associated with a sample, where the dataset includes quantitative data about the amounts of EVs or the level of the biomarkers associated therewith which have been found to be predictive of severity of NASH and/or liver fibrosis with a statistical significance less than 0.2 (e.g., p value less than about 0.05), and inputting the dataset into an analytical process that uses the dataset to generate a result useful in diagnosing and monitoring NAFLD, NASH, liver fibrosis, or other liver damage or diseases. In certain embodiments, the dataset also includes quantitative data about other clinical indicia or other marker associated with liver fibrosis.

Datasets containing quantitative data, typically the level/amount of EVs, or the levels of the biomarker associated therewith, and quantitative data for other dataset components can be inputted into an analytical process and used to generate a result. The analytical process may be any type of learning algorithm with defined parameters, or in other words, a predictive model. Predictive models can be developed for a variety of NAFLD classifications by applying learning algorithms to the appropriate type of reference or control data. Multivariable modeling can be applied to generate a risk score for diagnosing liver fibrosis. A risk score can be derived from the amount of total- or hepatocyte-derived EVs or the level of the biomarker of interest as determined by the methods described herein. The risk score can be compared to a control value, to provide information for diagnosing liver fibrosis in a subject. The result of the analytical process/predictive model can be used by an appropriate individual to take the appropriate course of action.

In certain embodiments, a scoring system or risk score can be generated by the analytical process to diagnose and monitor NAFLD, NASH, liver fibrosis, or other liver damage and diseases. In some aspects, the analytical process can use a dataset that includes the level/amount of total- or biomarker-associated EVs or the level of the biomarker of interest in a subject’s sample as determined by the methods described herein. The risk score can then be compared to a control value, to provide information for diagnosing or monitoring or assessing NASH and/or liver fibrosis or other liver damage or diseases in a subject.

In other aspects, the analytical process can use a reference dataset that includes the determined level/amount of EVs or the level of the biomarker of interest and quantitative data from one or more clinical indicia to generate a risk score. The risk score can be derived using an algorithm that weights the level/amount of biomarker-associated EVs or the level of the biomarker of interest in the sample and one more clinical indicia (or anthropometric features or measures) including but not limited to, age, gender, race, with or without diabetes, with or without hypertension, with or without hyper lipidemia, BMI, weight, height, waist circumference, hip/waste ratio, and other laboratory data including but not limited to aspartate aminotransferase (AST), alanine aminotransferase (ALT), AST/ ALT ratio, gamma GT, bilirubin, alkaline phosphatase, albumin, prothrombin time, platelet count, creatinine, total cholesterol, HDL, LDL, Triglycerides, triglyceride:HDL ratio, fasting glucose, fasting insulin, glucose/insulin ratio and Homeostatic Model Assessment index measuring insulin resistance. By way of example, a score derived from the formula: risk score = -10.051 + 0.0463*Age (year) + 0.147*BMI (kg/m²) + 0.0293*AST (IU/L) + 2.658*Total EVs (EV number/microliter).

In certain embodiments, the one or more clinical indicia can include at least one of the subject’s age, body mass index, or concentration of aspartate transaminase or alanine transaminase. In other embodiments, the one or more clinical indicia can include at least two of the subject’s age, body mass index, and concentration of aspartate transaminase or alanine transaminase. In other embodiments, the dataset can include the determined level/amount of biomarker-associated EVs or the level of the biomarker associated therewith, the subject’s age, body mass index, and concentration of aspartate transaminase or alanine transaminase.

The analytical process used to generate a risk score may be any type of process capable of providing a result useful for classifying a sample, for example, comparison of the obtained dataset with a reference dataset, a linear algorithm, a quadratic algorithm, a decision tree algorithm, or a voting algorithm. Prior to input into the analytical process, the data in each dataset can be collected by measuring the values for biomarker-associated EVs or the biomarkers associated therewith usually in triplicate or in multiple triplicates. The data may be manipulated, for example, raw data may be transformed using standard curves, and the average of triplicate measurements used to calculate the average and standard deviation for each patient. These values may be transformed before being used in the models, e.g. log-transformed or Box-Cox transformed. This data can then be input into the analytical process with defined parameters. The analytical process may set a threshold for determining the probability that a sample belongs to a given class. The probability preferably is at least 50%, at least 60%, at least 70%, at least 80%, at least 90%, or higher.

In certain embodiments, the analytical process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method. In some embodiments, the analytical process is based on a regression model, preferably a logistic regression model. Such a regression model includes a coefficient for biomarker-associated EVs or each of the biomarkers in a selected set of biomarkers disclosed herein. In such embodiments, the coefficients for the regression model are computed using, for example, a maximum likelihood approach. In particular embodiments, molecular marker data from the two groups (e.g., healthy and diseased) is used and the dependent variable is the status of the patient for which marker characteristic data are from.

By way of example, the analytical process can include a logistic regression model that generates a risk score based on the following algorithm: risk score = [-10.051+0.0463*Age(years)+0.147*BMI(kg/m²)+0.0293*AST(IU/L)+2.658*Total biomarker associated EVs (EV number/microliter)]*10) is determined. The risk score can be converted to a probability distribution with a value of 0 to 100 by the following algorithm; evLD=100*exp(z)/[I+exp(z)], wherein evLD is the probability distribution and z is the risk score calculated using the above noted algorithm.

It will be appreciated, that other analytical processes can be used to generate a risk score. These analytical processes can include for example a Linear Discriminant Analysis model, a support vector machine classification algorithm, a recursive feature elimination model, a prediction analysis of microarray model, a classification and regression tree (CART) algorithm, a FlexTree algorithm, a random forest algorithm, a multiple additive regression tree (MART) algorithm, or Machine Learning algorithms.

A risk score or result generated by the analytical process can be any type of information useful for making a LD classification, e.g., a classification, a continuous variable, or a vector. For example, the value of a continuous variable or vector may be used to determine the likelihood that a sample is associated with a particular classification.

LD classification refer to any type of information or the generation of any type of information associated with liver fibrosis, NAFLD, and/or NASH, for example, diagnosis, staging, assessing extent of liver fibrosis, NAFLD, and/or NASH, prognosis, monitoring, therapeutic response to treatments, screening to identify compounds that act via similar mechanisms as known NAFLD, NASH, and/or liver fibrosis treatments.

In some aspects, the result is used for diagnosis or detection of the occurrence of liver fibrosis. In this embodiment, a reference or training set containing “healthy” and “liver fibrosis (LF)” samples is used to develop a predictive model. A dataset, preferably containing level/amount of biomarker-associated EVs or the level of the biomarker associated therewith, indicative of NASH, is then inputted into the predictive model in order to generate a result. The result may classify the sample as either “healthy” or “LF” or staging of “LF”. In other embodiments, the result is a continuous variable providing information useful for classifying the sample, e.g., where a high value indicates a high probability of being a “LF” sample and a low value indicates a low probability of being a “healthy” sample.

In other embodiments, the result is used for liver fibrosis, NAFLD, and/or NASH, staging. In these embodiments, a reference or training dataset containing samples from individuals with disease at different stages is used to develop a predictive model. The model may be a simple comparison of an individual dataset against one or more datasets obtained from disease samples of known stage or a more complex multivariate classification model. In certain embodiments, inputting a dataset into the model will generate a result classifying the sample from which the dataset is generated as being at a specified NAFLD, NASH, and/or liver fibrosis disease stage. Similar methods may be used to provide NAFLD, NASH, and/or liver fibrosis prognosis, except that the reference or training set will include data obtained from individuals who develop disease and those who fail to develop disease at a later time.

In other embodiments, the result is used determine response to NAFLD, NASH, and/or liver fibrosis treatments. In this embodiment, the reference or training dataset and the predictive model is the same as that used to diagnose NAFLD, NASH, and/or liver fibrosis (samples of from individuals with disease and those without). However, instead of inputting a dataset composed of samples from individuals with an unknown diagnosis, the dataset is composed of individuals with known disease which have been administered a particular treatment and it is determined whether the samples trend toward or lie within a normal, healthy classification versus an NAFLD, NASH, and/or liver fibrosis classification.

In other embodiments, the result is used for drug screening, i.e., identifying new agents that normalize the presence of biomarker-associated EVs or the biomarker(s) associated therewith, so as to inhibit liver damage or hepatocyte lipotoxicity to angiogenesis and disease progression. Any drug screening methods now known or later developed in the art will be encompassed by the invention.

In other embodiments, the result is used for drug screening, i.e., identifying compounds that act via similar mechanisms as known NAFLD, NASH, and/or liver fibrosis drug treatments. In this embodiment, a reference or training set containing individuals treated with a known NAFLD, NASH, and/or liver fibrosis drug treatment and those not treated with the particular treatment can be used develop a predictive model. A dataset from individuals treated with a compound with an unknown mechanism is input into the model. If the result indicates that the sample can be classified as coming from a subject dosed with a known NAFLD, NASH, and/or liver fibrosis drug treatment, then the new compound is likely to act via the same mechanism.

One of skill will also recognize that the results generated using these methods can be used in conjunction with any number of the various other methods known to those of skill in the art for diagnosing and monitoring NAFLD, NASH, liver fibrosis, or other liver damage or diseases.

Using methods described herein, skilled physicians may select and prescribe treatments adapted to each individual subject based on the diagnosis of NAFLD, NASH, and/or liver fibrosis provided to the subject through determination of the level/amount of EVs or the level of the biomarkers associated therewith in a subject’s sample. In particular, the present invention provides physicians with a non- subjective means to diagnose NAFLD, NASH, and/or liver fibrosis, which will allow for early treatment, when intervention is likely to have its greatest effect. Selection of an appropriate therapeutic regimen for a given patient may be made based solely on the diagnosis provided by the inventive methods. Alternatively, the physician may also consider other clinical or pathological parameters used in existing methods to diagnose NAFLD, NASH, and/or liver fibrosis and assess its advancement.

The invention further provides a method of treating NAFLD, NASH, liver fibrosis or other associated liver damage or diseases using any drugs, compounds, small molecules, proteins, antibodies, nucleotides, and pharmaceutical compositions thereof, that are capable of normalizing levels circulating biomarker-associated EVs and/or biomarker(s) associated therewith.

The invention contemplates any conventional methods for formulation of pharmaceutical compositions as described above. Various additives, known to those skilled in the art, may be included in the formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

Effective dosages and administration regimens can be readily determined by good medical practice and the clinical condition of the individual subject. The frequency of administration will depend on the pharmacokinetic parameters of the active ingredient(s) and the route of administration. The optimal pharmaceutical formulation can be determined depending upon the route of administration and desired dosage. Such formulations may influence the physical state, stability, rate of in vivo release, and rate of in vivo clearance of the administered compounds.

Depending on the route of administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug’s specific activity, the severity of the damage and the responsiveness of the patient, the age, condition, body weight, sex and diet of the patient, the severity of any present infection, time of administration and other clinical factors.

The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

It is to be noted that throughout this application various publications and patents are cited. The disclosures of these publications are hereby incorporated by reference in their entireties into this application in order to describe fully the state of the art to which this invention pertains.

### EXAMPLES

### Results

### Isolation and Characterization of Plasma EVs

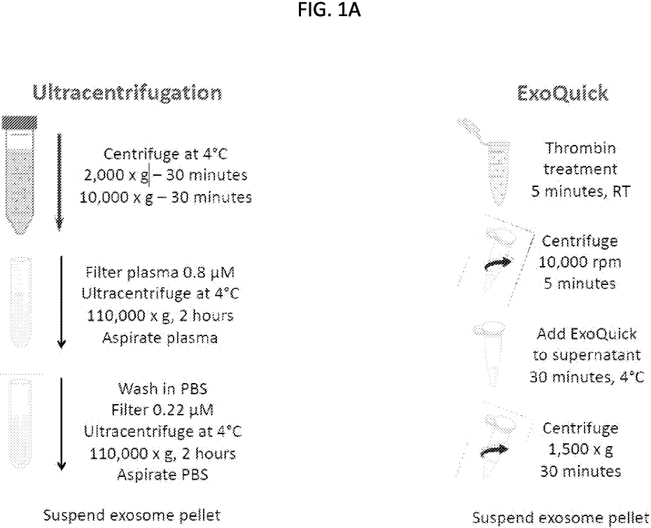

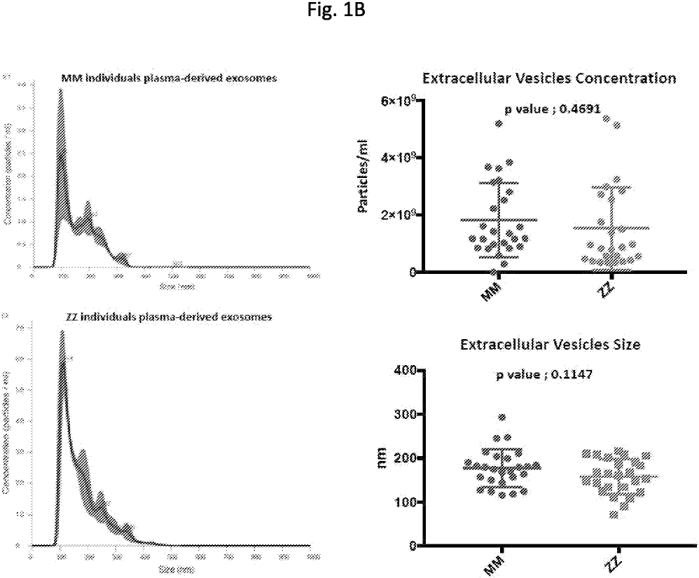

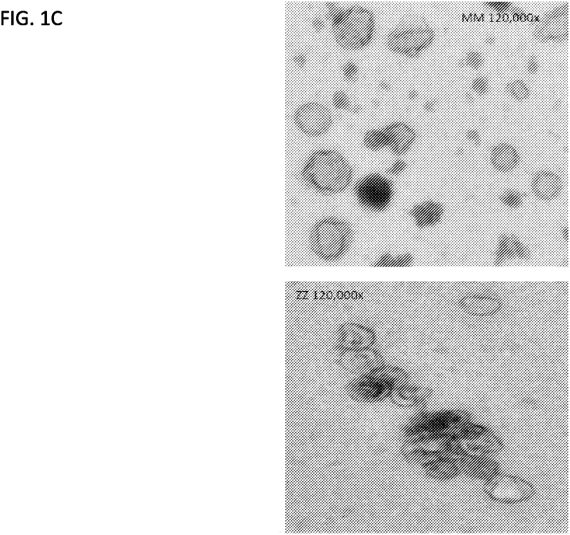

EVs were isolated from plasma of control subjects (MM) and AATD individuals (ZZ) by a combination of filtration and ultracentrifugation (FIG. 1A). Particles purified from plasma were characterized by analyses for particle size, distribution, and concentration using Nanoparticle tracking analysis (NTA) and transmission electron microscopy (TEM). The plasma-derived EVs were within the normal range for EV size (30-200 nm in diameter) (FIG. 1B). Negative staining of classic TEM demonstrated cup-shaped, round particles (FIG. 1C). The presence of EV markers was also investigated by western blot analysis. The results confirmed abundant CD63, CD81, and TSG101 expression in all EV fractions. Among detected EV protein markers, TSG101 was enriched in ZZ plasma derived-EVs compared to MM healthy controls (FIG. 1D).

### Comparison of EV-Associated and Plasma Cytokines and Chemokines Between Control and AATD Subjects

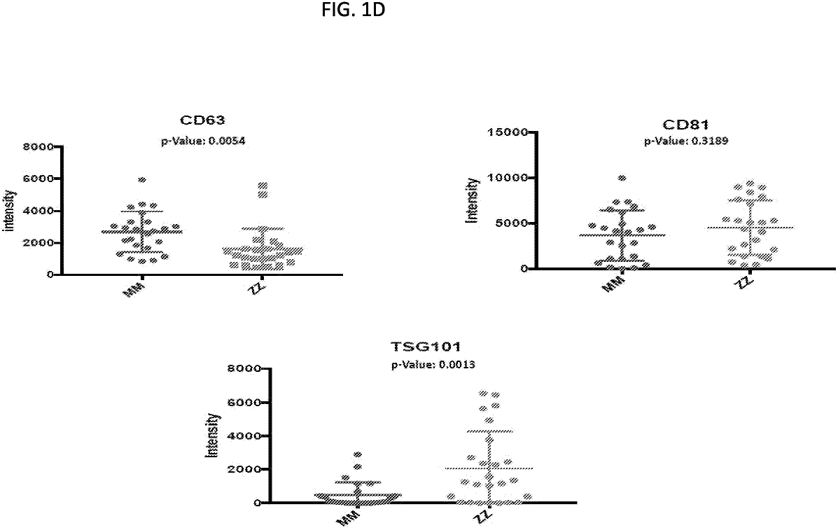

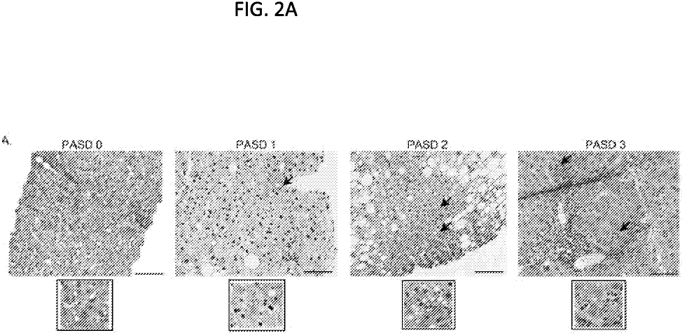

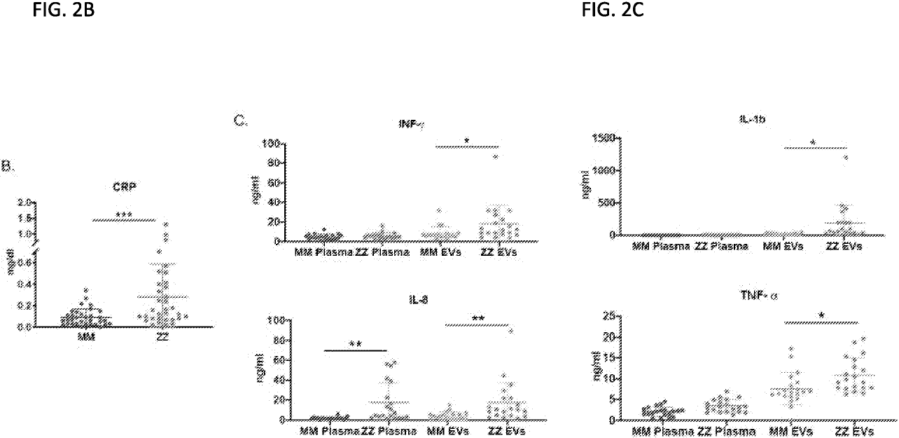

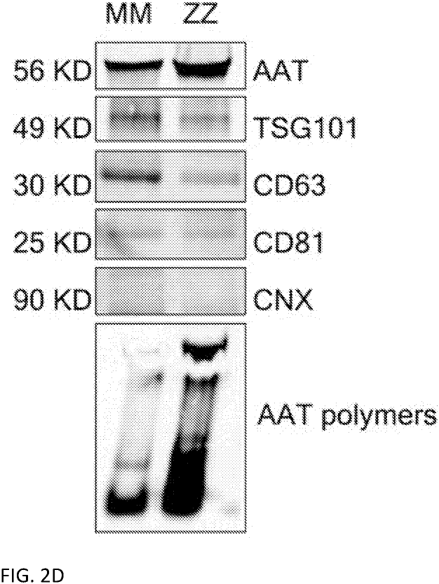

We evaluated 26 plasma samples in each group of samples. The diagnosis of AATD individuals was confirmed. Phenotyping was by isoelectric focusing. Although liver biopsy is not required for diagnosis, it may be helpful in difficult cases and/or for prognosis of liver disease. Histologically, insoluble ZAAT proteins are characterized by globules which stain bright pink with periodic acid-Schiff’s reagent, which are resistant to diastase treatment (PAS+D) (FIG. 2A). PAS+D globules present in a high-power field were scored from 0-3 as follows: 0-None, 1-Rare: <5 hepatocytes with globules, 2-Few: 5-20 hepatocytes with globules, 3-Numerous: ≥20 hepatocytes with globules (13). We measured CRP levels of healthy control and AATD individuals. The AATD individuals were divided into normal (<0.4 mg/dL) and elevated (≥0.4 mg/dL) CRP levels. Approximately one-third of patients (32%) had CRP levels ≥0.4 mg/dL, indicative of inflammation. All healthy controls (100%) had CRP levels (<0.4 mg/dL), indicative of a healthy condition with no sign of inflammation (FIG. 2B). Then, we evaluated the absolute amounts of each cytokine in the EV fraction versus the un-encapsulated (soluble) form from each sample. Distribution of each cytokine between the free and EV-encapsulated form is a characteristic of a system, rather than of the cytokines being secreted by the same pathway in all systems (22). We found that in our study groups, the levels of EV-associated INF-γ, IL1-β, and TNF-α cytokines were significantly higher in AATD individuals, whereas there was no difference between the levels of free form of the same cytokines in the control group. The level of both free form and EV-associated IL-8 was significantly higher in AATD individuals (FIG. 2C). Then, utilizing western blot analysis we evaluated the presence of AAT protein in the plasma derived EVs pooled from 4 MM and four ZZ individuals with PAS + D scores of 3 and found that the total levels of AAT is higher in EVs isolated from ZZ individuals. Furthermore, non-denaturing western blot analysis showed that ZZ plasma derived EVs carry significant amounts of AAT aggregates (FIG. 2D).

### Differential miRNA Expression Profiles Among Plasma Derived EV miRNAs from Healthy and AATD Individuals

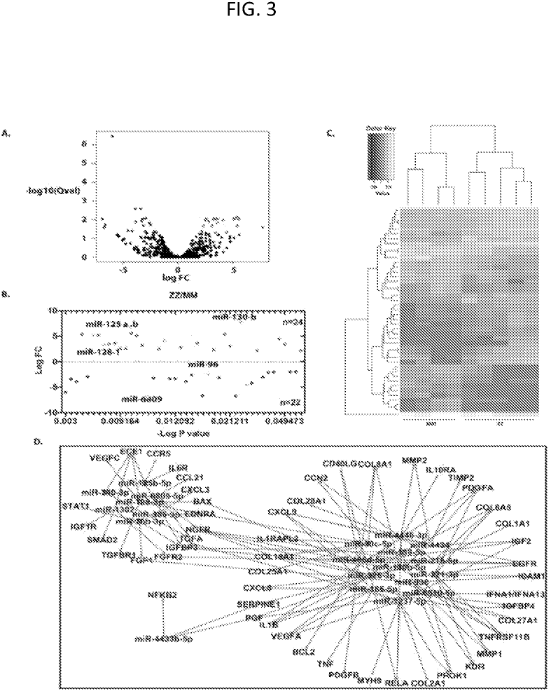

To explore the expression profiles of miRNAs in plasma-derived EVs from MM and ZZ individuals, we isolated EVs as previously described. The EVs were then validated by NTA and electron microscopy analyses. We then performed high-throughput sequencing of small-RNA transcriptomes (≤50 nt) for miRNA expression profiles. A total of 869 miRNAs in MM and ZZ groups were identified at the threshold of > 1 CPM (Counts Per Million) in at least half of the samples and the whole distribution of differentially expressed miRNAs was visualized in the volcano plots (FIG. 3A). We identified 44 differentially expressed miRNAs at thresholds of fold change > 2 and q value < 0.05 in the plasma derived EVs from the healthy MM individuals compared with ZZ individuals. Among the differentially expressed miRNAs, 24 were upregulated and 22 were downregulated (FIG. 3B) in the EVs of the ZZ individuals. The top 10 differentially expressed miRNAs were the hsa-miR-125 family, hsa-miR-335-3p, hsa-miR-339-5p, hsa-miR-4433b-5p, hsa-miR-130b-5p, hsa-miR-658, hsa-miR-6809, and hsa-miR-6510-5p (Table 1). A heatmap of differentially expressed miRNAs where gray and green indicate significantly expressed miRNAs is shown in FIG. 3C. The results of network analysis are presented in FIG. 3D. Interestingly, the analysis revealed molecular networks targeted by miRNAs involved in HSCs activation. The networks contained genes predicted to be involved in ECM synthesis, Inflammatory pathways and cellular growth and proliferation.

### Plasma Derived-EVs From AATD Individuals Activate Hepatic Stellate Cells in Vitro

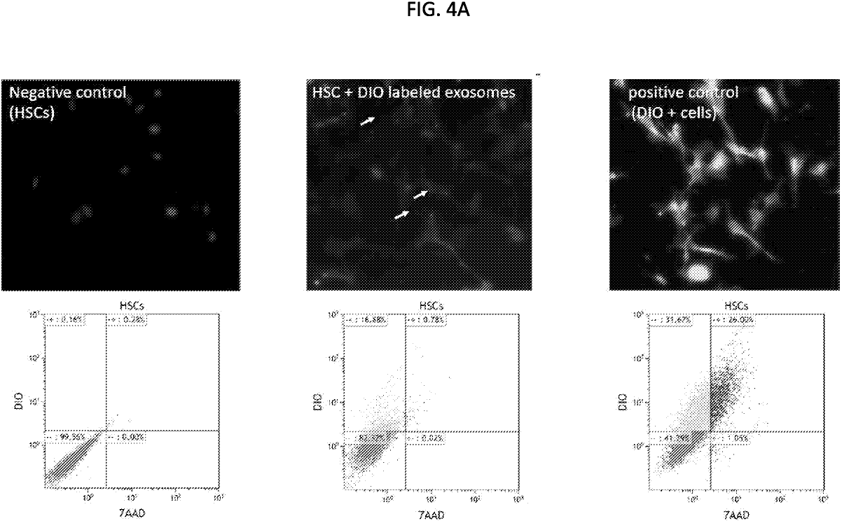

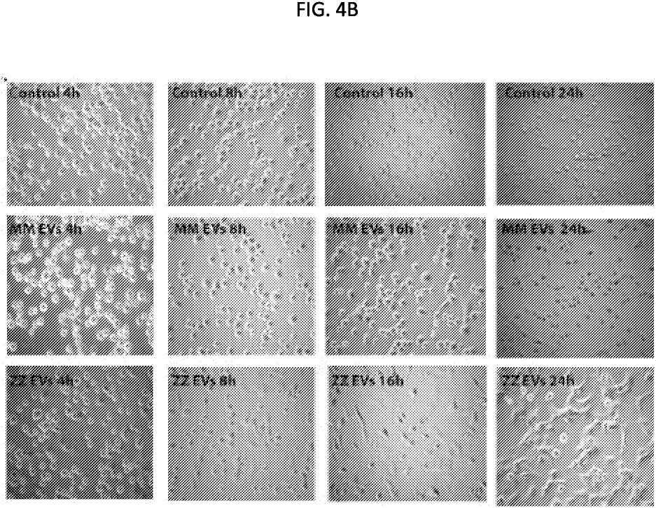

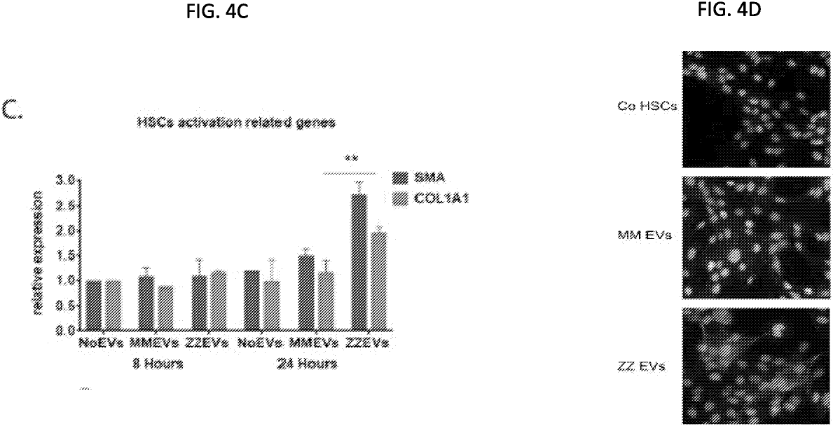

In order to evaluate the interaction and possible uptake of plasma derived-EVs by cultured LX-2 cells, purified EVs were labeled with DiO and incubated with the HSCs for fluorescence microscopy and flow cytometry analysis. Fluorescence microscopy images of the treated cells exhibited green spots or small patches. In contrast, the HSCs treated with DiO alone showed a homogeneous green fluorescent staining. HSCs incubated with DiO-labeled EVs were also analyzed by flow cytometry to provide quantitative measurement of the above internalization. HSCs treated with DiO-labeled EVs produced lower fluorescent intensity compared to cells stained with DiO only (FIG. 4A). Residual dye was substantially eliminated after a second wash, suggesting that the signal from DiO EVs was from the DiO associated with EVs. To further evaluate the bioactivity of the plasma-derived EVs, quiescent LX-2 cells and iPSc-derived HSCs were incubated with or without MM or ZZ plasma-derived EVs for 24 hours and cell morphology was monitored by light microscopy. Images taken from different treatment conditions showed that quiescent iPSc-derived HSCs incubated with ZZ plasma-derived EVs exhibited an elongated, fibroblast-like shape after 8 hours, indicating an activated state. In contrast, the iPSc- derived HSCs incubated without EVs or with MM plasma-derived EVs exhibited a more rounded morphology suggestive of a more quiescent state (FIG. 4B). The activation status of HSCs was determined by quantifying α-SMA and Col1 A1 mRNA expression, markers of stellate cell activation (23). They were found to be significantly altered by incubation with ZZ plasma-derived (FIG. 4C). Next, we investigated the effect of ZZ plasma-derived EVs on the protein levels of αSMA in HSCs. Consistent with our findings above, only cells incubated with ZZ plasma-derived EVs had significant increase of α-SMA green fluorescent signal compared to controls (FIG. 4D).

### NF-κB and JAK/STAT-Dependent Regulation of Activation and Migration of HSCs

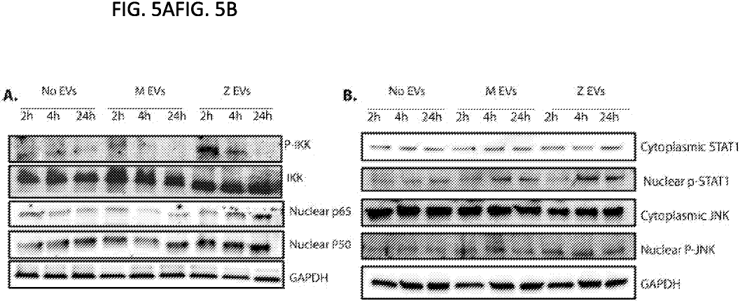

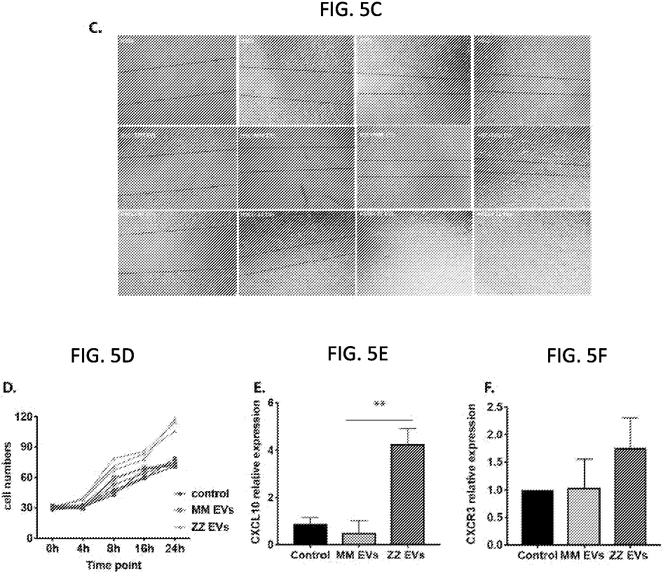

Previous studies have demonstrated that inflammatory signaling pathways such as NF-κB and JAK/STAT significantly promote trans differentiation and activation in HSCs at the early stages of inflammation (24-27). To investigate the signaling pathways likely to be responsible for HSC activation by ZZ plasma-derived EVs, we quantified the signaling molecules involved in the canonical NF-κB and JAK/STAT signaling pathways in iPSC-derived HSCs treated with or without plasma-derived EVs. Phosphorylation of IKK, which is central to the NF-κB signaling pathway, and nuclear translocation of p50 and p65 subunits, which are involved in the transcription (28), were all significantly increased in iPSC-derived HSCs treated with ZZ plasma-derived EVs compared to controls (FIG. 5A). The phosphorylation of nuclear STAT1 and JNK were also slightly elevated in iPSC-derived HSCs treated with ZZ plasma-derived EVs compared to controls (FIG. 5B). Thus, ZZ plasma-derived EVs regulate the transition process of HSCs from a quiescent to activated state through canonical NF-κB and JAK/STAT signaling pathways. Acquiring migratory and contractile capacities are also very important properties of activated HSCs, so we investigated the effects of plasma-derived EVs on these properties. Wound healing assays and cell counting revealed that ZZ plasma-derived EVs significantly enhanced the migratory and contractile capacities of LX2 cells (FIG. 5C and D). Furthermore, HSCs migration and proliferation require stimulation of the CXCR3 receptor on HSCs with the CXCL10 ligand (29,30). Therefore, we detected the over expression of CXCL10 mRNA, which is downstream of the NF-κB signaling pathway, to confirm migratory augmentation of HSCs after 24 hours of incubation with ZZ plasma-derived EVs by qPCR (FIG. 5E). We have also confirmed the expression of CXCR3 receptor in our HSC model (FIG. 5F).

### High CXCL10 Plasma Levels Are Associated With AATD-Mediated Liver Fibrosis in AATD Individuals

Previous observation suggests that the CXCR3-associated chemokines, particularly CXCL10, play an important role in the development of inflammation and fibrosis in the liver parenchyma (31). It has been shown that the IFN-γ inducible chemokines CXCL9, CXCL10, and CXCL11 are up-regulated in patients with chronic liver diseases, and their serum levels are closely associated with the degree of hepatic fibrosis. Among these chemokines which share the receptor CXCR3, CXCL10 appears to be profibrogenic by direct effects on HSCs (32). Within the liver, hepatic stellate cells express CXCR3 which promotes HSC migration through interaction with CXCL10 ligand (30). To further identify the potential association between CXCR3-associated CXCL10 and HSC activation and migration, we evaluated the plasma levels of CXCL10 in samples obtained from 44 ZZ individuals and 36 healthy MM controls. We observed that the mean CXCL10 plasma level was significantly higher among the ZZ individuals compared to the healthy MM group (FIG. 6A). Among the ZZ individuals, we quantified the association between activated HSCs by investigating intrahepatic α-SMA positive cells and the plasma levels of CXCL10 in ZZ individuals from whom biopsy samples were available. As expected, we observed that the numbers of intrahepatic α-SMA positive cells is corelated to the plasma levels of CXCL10 (FIG. 6B). Immunohistochemical staining of the liver biopsies from ZZ individuals demonstrated that those with higher plasma levels of CXCL10 had substantially greater intrahepatic α-SMA positive cells compared to those with lower plasma levels (FIG. 6C).

### High Liver Derived Exosomes Are Associated With More Liver AAT Accumulation in AATD Individuals

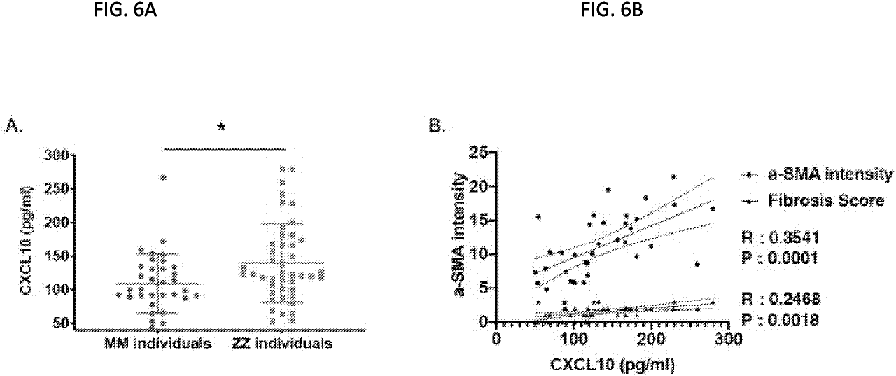

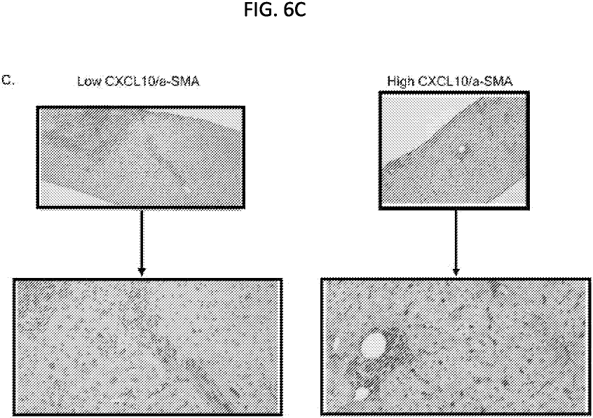

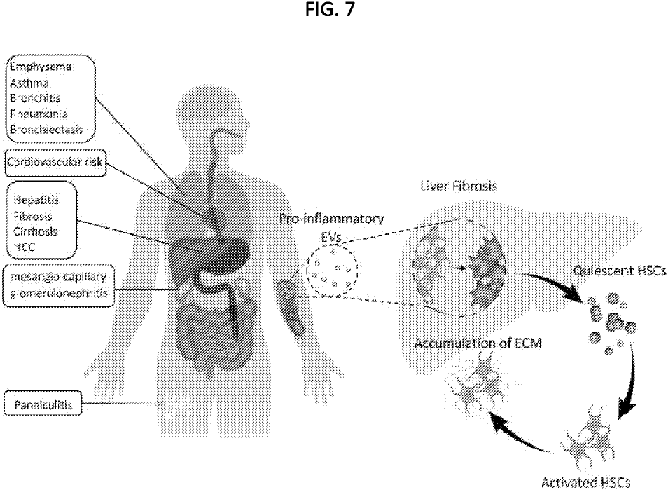

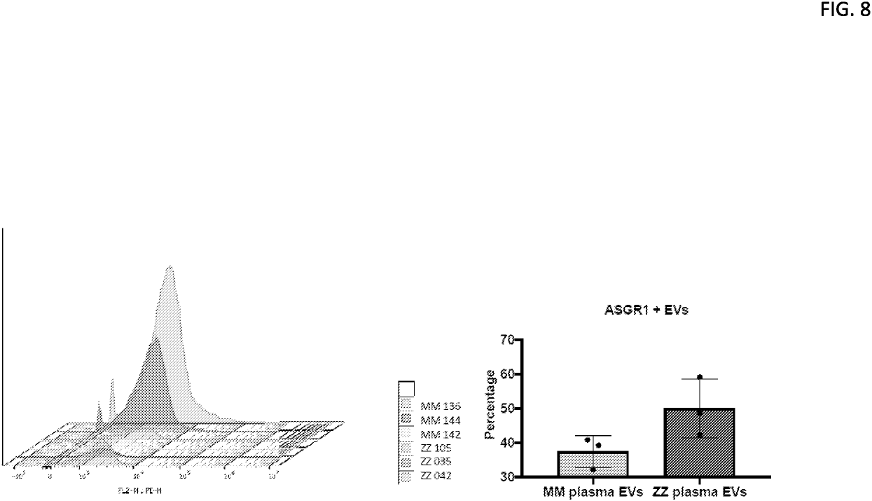

Exosomes secreted by the liver contain the transmembrane protein Asialoglycoprotein receptor 1 (ASGR1). EVs were isolated from plasma of control subjects (MM) and AATD individuals (ZZ) by a combination of filtration and ultracentrifugation. The exosomes comprising ASGR1 were identified using violet laser equipped CytoFlex flowcytometry. In healthy controls, approximately 30-40% of plasma derived exosomes originated from the liver. In AATD subjects, a greater number of the plasma derived exosomes originated from the liver with about 60% of the total plasma derived exosomes originating from the liver (FIG. 7). These data also indicate that patients with more liver AAT accumulation have more liver derived exosomes circulating compare to healthy subjects which have very low levels of liver AAT accumulation.

### Methods

### Clinical Samples

Subjects with an MM genotype were selected from the Alpha-1 Foundation DNA and Tissue Bank for use as a control group. Study subjects were chosen from a trial based at the University of Florida that followed patients with AATD over a 3-year period to evaluate progression of liver disease. Plasma from subjects with a ZZ genotype and liver biopsies indicating presence of fibrosis and high levels of PASD positive cells were used.

### Cell Culture

Human LX-2 cell line was purchased from MilliporeSigma (Burlington, MA) and were cultured in Dulbecco’s Modified Eagle’s Medium (DMEM) supplemented with 2% (v/v) fetal bovine serum and 1% primocinTM at 37° C. in a humidified incubator under 5% CO2. iPSc-derived HSCs were differentiated from induced pluripotent stem cells (iPScs) based on an established protocol (66). Briefly PBMC-derived undifferentiated iPScs were incubated with BMP4 for 4 days to induce mesodermal progenitors. Following this step, the cells were subsequently incubated with BMP4, FGF1, and FGF3 until day 6 to induce a liver mesenchymal submesothelial phenotype. From days 6 to 8, the cells were incubated with FGF1, FGF3, retinol, and palmitic acid. The final step of differentiation from submesothelium-like cells to HSCs was modeled by incubation of the cells with retinol and palmitic acid from day 8 to day 12. We next characterized differentiated iPSc- HSCs on day 12. iPSc-HSCs were analyzed for the expression levels of markers associated with HSCs including TIMP (Tissue Inhibitor of Metallopeptidase Inhibitor 1), CoL1A1 (Collagen type1 Alpha1), α-SMA (Alpha Smooth Muscle Actin) and LRAT (Lecithin: Retinol Acyl Transferase).

### Isolation of EVs

EVs were isolated from approximately 3 mL of cryopreserved plasma by differential centrifugation (67). Briefly, the plasma was diluted in PBS (1:1) and centrifuged at 2,000 x g for 30 minutes at 4° C. and at 12,000 rpm for 45 minutes at 4° C. Supernatants were collected into ultracentrifuge tubes and centrifuged in a Beckman SW-40Ti swinging rotor ultracentrifuge (Beckman Coulter) at 110,000 x g for 2 hours at 4° C. Resuspended pellets in PBS were filtered through a 0.22-µm filter (Millipore, Billerica, MA) and centrifuged at 110,000 × g for 70 minutes at 4° C. EV pellets were washed with PBS and centrifuged at 110,000 × g for 70 minutes at 4° C. and resuspended in 200 µL of PBS. EV preparations were conserved at -80° C. for later use.

### Particle Number and Size Measurement

The size distribution and concentration of the isolated EVs was analyzed by Nanosight NS300 system (Malvern Instruments Company, Nanosight, and Malvern, United Kingdom). Briefly, purified EVs were homogenized by vortexing followed by dilution of 1:100 in sterile PBS and analyzed by Nanosight NS300. Each sample analysis was conducted for 60 seconds. Data was analyzed by Nanosight NTA 2.3 Analytical Software (Malvern Instruments Company, Nano sight, and Malvern, United Kingdom) with the detection threshold optimized for each sample and screen gain at 10 to track as many particles as possible with minimal background. A blank 0.2 µm- filtered 1x PBS was also run as a negative control. At least five analysis were done for each individual sample.

### Transmission Electron Microscopy

Purified EVs were fixed with 2% paraformaldehyde. A 20 µl drop of the suspension was loaded onto a formvar coated grid, negatively stained with 2% aqueous uranyl acetate for 2 minutes, and examined under a Hitachi 7600 transmission electron microscope (Hitachi High-Technologies America, Schaumburg, IL) equipped with a Macrofire monochrome progressive scan CCD camera (Optronics, Goleta, CA) and AMTV image capture software (Advanced Microscopy Techniques, Danvers, MA).

### EVs RNA Isolation, Small RNA Library Preparation, Sequencing, and Bioinformatics

RNA was isolated using exoRNeasy Serum/Plasma Maxi kit (Qiagen) according to the manufacturer’s instructions with the final elution volume of 12 µl from purified EV fractions derived from 4 MM healthy plasma and 5 ZZ plasma samples. The quantity and quality of the RNA were determined using the Agilent RNA 6000 Pico Kit to determine the concentration of total RNA, and a Small RNA Kit Chip was used to measure the concentration of EV micro RNA (miRNA) on the Agilent Bioanalyzer instrument (Agilent Technologies). Total RNA samples contained a range of 50-90% miRNA. Sequencing libraries were constructed using ~2 ng of total EV RNA with Small RNA Library Prep Set for Illumina kit (New England BioLabs) and were sequenced using Illumina Miseq 1x150 cycles V3 kit at the University of Florida Interdisciplinary Center for Biotechnology Research. The expression of the miRNAs was obtained using miRDeep2 software (68) and differential analysis was performed using the exact test from edgeR package (69). We performed enrichment analysis using Ingenuity Pathway Analysis (IPA) (Ingenuity Pathways Analysis (IPA) system. Redwood, CA: Ingenuity Systems, Inc;) package using the miRNA-mRNA target link module to link the discovered significantly differentially expressed miRNAs with their mRNA target genes. The IPA miRNA-mRNA target link module derives information from TargetScan (TargetScan Human Prediction of miRNA targets. Release 7.1. Jun, 2016.), a database containing miRNAs and their predicted target genes, along with prediction scores and the experimental conformation from the literature. Because IPA does not include prediction scores, to validate the miRNA target genes, we performed additional computational analysis using TargetScan to identify target genes with good predicted scores. In addition, we used IPA to identify miRNA-mRNA target genes which have been experimentally confirmed.

### Cytokine Measurement

We first identified priority cytokine candidates based on known liver fibrosis pathophysiology and previously published literature, then selected from the assays available at Myriad-RBM, which were primarily multiplexes (Luminex xMap technology, Myriad-RBM Inc., Austin TX). Each multiplex measured a number of analytes in addition to the priority cytokines. Then purified EVs from 26 MM and 26 ZZ plasma samples, were suspended in a mixture of water and PBS (1:2). EVs in solution were lysed using an equal volume of IP lysis buffer. Lysed EV samples to which Triton X was added at final concentration of 1% were run on the MilliPLEX Human High Sensitivity T Cell Magnetic Bead Panel Luminex kit for measurement of 21 unique cytokines per plex according to manufacturer’s instructions (EMD Millipore, St. Louis, MO). The Luminex® 200™ analyzer, which measures multiple analytes in a 50 □L aliquot of processed EVs was used to analyze the cytokine profiles. This system is a dual laser, flow-based sorting and detection platform, which uses analyte-specific antibodies pre-coated onto color-coded microparticles to measure 1-25 possible cytokines. Microparticles are read using the Luminex 200 analyzer. One laser is microparticle-specific and determines which cytokine is being detected. The other laser determines the magnitude of the phycoerythrin-derived signal, which is in direct proportion to the amount of analyte bound. The individual cytokines are quantified against an 8-point calibration curve run with each plate.

### Labeling of EVs and Uptake

EVs were obtained as described in the “Isolation of EVs” section and were labeled with lipophilic green fluorescent dye (DiO) and incubated with the LX2 cells for fluorescence microscopy study and flow-cytometry analysis. For labeling with DiO, purified EVs from plasma (109 CD63-positive particles) were incubated with Fast DiO green fluorescent membrane dye (Invitrogen) at a final concentration of 2 µg/mL for 1 hour at room temperature. Labeled EVs were diluted with PBS and spun at 120,000 × g for 90 minutes to sediment labeled EVs and remove unbound dye. The purification process of washing and ultracentrifugation was repeated twice before the labeled EV pellet was resuspended in PBS (70). For microscopic analysis, LX2 cells were incubated with DiO-labeled EVs for 1 hour at 37° C. After incubation, cells were washed with PBS to remove unbound labeled EVs and subsequently imaged with a Keyence fully motorized BZ-X800 microscope (KEYENCE America, Chicago, USA).

For flow cytometry analysis, LX2 cells were seeded in six-well plates and grown overnight. Prior to treatment with DiO-labeled EVs, cells were washed with PBS, and then DiO-labeled EVs (2×107) in 100 µL PBS/well (CD63-positive) were added and incubated at 37° C. for 1 hour. Cells stained directly with 1 µL/well DiO (1 µg/mL) served as a positive control, and unstained cells as a negative control. Cells were removed from plastic by trypsin, centrifuged, and resuspended in 500 µL PBS. Following staining, the cells were analyzed using a Beckman Coulter Gallios Flow Cytometer using Kaluza acquisition software and Flow Jo for analysis. Each experimental group was performed in triplicate.

### Co-Incubation of Human Plasma-Derived EVs With Hepatic Stellate Cells

Hepatic stellate cells were plated at the cell concentration of 200,000 per well in 12-well plates. Isolated EVs (pooled from 5 control or AATD individuals (isolated from 250 □L plasma)) were added to each well and the plate was incubated for 48 h at 37° C. (67). HSCs were incubated with the same volume of PBS and have been used as negative control.

### Quantitative Real-Time PCR

Total RNA was extracted using Trizol reagent (Takara, A7603-1), and reversely transcribed through SuperScript VILOTM kit (Invitrogen, Carlsbad, CA) according to the protocol. Real-time PCR analyses were performed with SensiFAST qPCR Master Mix (Bioline, Memphis, TN) on a 7500 Real-time PCR system, Applied Biosystems, at the recommended thermal cycling settings: one initial cycle at 95° C. for 30s followed by 40 cycles of 5 s at 95° C. and 30s at 60° C. Probes used for human Acta2, Col1 a1, Timp1 and LRAT detection were purchased from Thermofisher (Carlsbad, CA).

### Immunoblotting

Human LX-2 cell line and iPSc-derived HSCs were seeded at 3 x 105/well in 6-well plates with or without MM or ZZ plasma-derived EVs for 24 hours. Then, RIPA buffer was used to lyse the cells. Protein levels in the cell lysate homogenates were determined using the bicinchoninic acid (BCA) method (Pierce Biotechnology, Rockford, IL). Total protein was resolved on tris glycine SDS- PAGE gels (Bio-Rad). Proteins were transferred to nitrocellulose membranes. The blots were incubated with rabbit polyclonal antibodies against total and phospho-IKK, p65, p50, total and phospho-STAT, total and phosphor-JNK and GAPDH (Cell Signaling, Danvers, MA), CD81, CD63 and TSG101 (Proteintech, Chicago, IL) overnight at 4° C. after blocking. Proteins were detected by using a Super Signal West Dura Extended Duration Substrate Kit from Thermo Scientific. Western blot band intensities were quantified using Alpha view software (ProteinSimple, San Jose, CA).

### Immunostaining and Immunofluorescence Microscopy

Human LX-2 cell line treated with or without MM or ZZ plasma-derived EVs were grown on glass coverslips. After 24 hours, the cells were fixed with 4% paraformaldehyde in PBS for 20 minutes. The coverslips were washed with 1X PBS. The cells were incubated for 1 hour with blocking buffer (1X PBS, 5% goat serum, 0.3% Triton X-100) at room temperature, followed by incubating overnight at 4° C. with primary antibodies (1 :400). The cells were washed with 1X PBS and incubated for 1 hour with secondary antibodies (Alexa Fluor 488 goat anti-mouse IgG and Alexa Fluor 594 goat anti-rabbit IgG). The coverslips were mounted and sealed. Images were collected using a Keyence fluorescence microscope (Osaka, Japan). Samples were scanned with a 0.1- mm step. Images were processed for brightness and contrast and filtered for noise following good practices as outlined by Rossner and Yamada (71).

### Liver Histology

A percutaneous liver biopsy was performed with a 16 gauge BioPince® core biopsy needle after using ultrasonography to mark the location. The sample was fixed in formalin and processed for examination. Stains included H&E, trichrome, PAS/PAS + D and Prussian blue. Portal inflammation and hepatocyte degeneration were noted. The presence of definite non-alcoholic steatohepatitis (NASH) was based on pathologist interpretation of hepatocyte ballooning, lobular inflammation, and steatosis (13).

### Statistical Analysis

All results are expressed as mean ± S.E. Statistical analyses were performed using Prism 8 software program (GraphPad Software) by Student t-test or Mann-Whitney U test. Values of P <0.05 was considered statistically significant.

TABLE 1

Significantly dysregulated miRNAs in ZZ plasma-derived EVs

| Up-regulated | Fold change | Down-regulated | Fold change |
|---|---|---|---|
| hsa-miR-125a-5p.hsa-mir-125a | 5.36697164 | hsa-miR-658.hsa-mir-658 | -5.9859744 |
| hsa-miR-125b-5p.hsa-mir-125b-1 | 5.18329317 | hsa-miR-4488.hsa-mir-4488 | -3.378354 |
| hsa-miR-125b-5p.hsa-mir-125b-2 | 5.18340772 | hsa-miR-4800-5p.hsa-mir-4800 | -3.8837823 |
| hsa-miR-139-5p.hsa-mir-139 | 3.30934504 | hsa-miR-324-5p.hsa-mir-324 | -3.1512944 |
| hsa-miR-151a-5p.hsa-mir-151a | 3.45601808 | hsa-miR-3656.hsa-mir-3656 | -2.9581834 |
| hsa-miR-151b.hsa-mir-151b | 3.61474378 | hsa-miR-6809-5p.hsa-mir-6809 | -6.8978544 |
| hsa-miR-30c-5p.hsa-mir-30c-1 | 2.59281873 | hsa-miR-4516.hsa-mir-4516 | -3.2291639 |
| hsa-miR-30c-5p.hsa-mir-30c-2 | 2.59370236 | hsa-miR-218-5p.hsa-mir-218-1 | -3.3050034 |
| hsa-miR-335-3p.hsa-mir-335 | 5.55148985 | hsa-miR-218-5p.hsa-mir-218-2 | -3.3052121 |
| hsa-miR-339-5p.hsa-mir-339 | 5.07518785 | hsa-miR-6867-5p.hsa-mir-6867 | -3.8457938 |
| hsa-miR-340-3p.hsa-mir-340 | 3.22016136 | hsa-miR-451a.hsa-mir-451a | -2.6098699 |
| hsa-miR-128-3p.hsa-mir-128-1 | 2.5067686 | hsa-miR-6510-5p.hsa-mir-6510 | -6.6429613 |
| hsa-miR-328-3p.hsa-mir-328 | 4.1125644 | hsa-miR-96-5p.hsa-mir-96 | -2.506894 |
| hsa-miR-99a-5p.hsa-mir-99a | 2.78701394 | hsa-miR-936.hsa-mir-936 | -6.7286907 |
| hsa-miR-191-3p.hsa-mir-191 | 3.57966715 | hsa-miR-16-5p.hsa-mir-16-2 | -1.9759375 |
| hsa-miR-128-3p.hsa-mir-128-2 | 2.29874622 | hsa-miR-4793-5p.hsa-mir-4793 | -4.5613285 |
| hsa-miR-4433b-5p.hsa-mir-4433b | 5.40370557 | hsa-miR-4298.hsa-mir-4298 | -4.1782314 |
| hsa-miR-4446-3p.hsa-mir-4446 | 3.07252979 | hsa-miR-16-5p.hsa-mir-16-1 | -1.9774818 |
| hsa-miR-130b-5p.hsa-mir-130b | 7.72943936 | hsa-miR-4306.hsa-mir-4306 | -2.9663986 |
| hsa-miR-222-3p.hsa-mir-222 | 2.17946999 | hsa-miR-15b-5p.hsa-mir-15b | -2.1452906 |
| hsa-miR-26b-3p.hsa-mir-26b | 4.44725049 | hsa-miR-106b-5p.hsa-mir-106b | -2.0233933 |
| hsa-miR-150-5p.hsa-mir-150 | 3.4166121 | | |
| hsa-miR-3615.hsa-mir-3615 | 2.17274498 | | |

Table 1. Positive and negative values for Log2(FC) represent miRNAs up-regulated or down-regulated, respectively, in ZZ individuals.

The teachings of any references cited herein are incorporated in their entirety to the extent not inconsistent with the present disclosure. Although the present invention has been described in considerable detail with reference to certain preferred versions thereof, other versions are possible. Therefore, the spirit and scope of the appended claims should not be limited to the description of the preferred versions contained herein. The reader’s attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state“means for” performing a specified function, or “step for” performing a specific function, is not to be interpreted as a “means” or “step” clause as specified in 35 U.S.C § 112, sixth paragraph. In particular, the use of step of in the claims herein is not intended to invoke the provisions of 35 U.S.C § 112, sixth paragraph.

What is claimed is:

1. A method of detecting liver disease in a subject, comprising: a) obtaining a biological sample of the subject, and b) analyzing the biological sample to determine a level(s) of one or more biomarkers indicative of liver disease, wherein the biological sample is blood, plasma, serum or circulating extracellular vesicles (EVs), and optionally administering a liver disease therapy.

2. The method of claim 1, wherein said circulating EVs are derived from hepatocytes.

3. The method of claims 1 or 2, wherein the one or more biomarkers comprise a biomolecule associated with the circulating EVs.

4. The method of any of claims 1-3, wherein the one or more biomarkers comprise at least one biomarker in free form.

5. The method of any of claims 1-4, wherein determining the level(s) of the one or more biomarkers comprises determining a level of at least one biomolecule that is associated with the circulating EVs.

6. The method of any of claims 1-3, wherein the one or more biomarkers comprise circulating EVs that have one or more biomolecules associated therewith and wherein analyzing the biological sample comprises determining the level of the circulating EVs that have one or more biomolecules associated therewith.

7. The method of any of claims 3-7, wherein the biomolecule comprises at least one biomolecule selected from the group consisting of TSG101, CXCL10, INF-γ, IL1-β, TNF-α, IL-8, AAT, ASGR1 and any miRNA or group of miRNAs set forth in Table 1.

8. The method of claim 7, wherein the biomolecule is CXCL10 and/or IL-8, and analyzing comprises determining the level of free-form CXCL10 and/or IL-8 in blood, plasma or serum.

9. The method of claim 7, wherein the biomolecule comprises TSG101, INF-γ, IL1-β, TNF-a, IL-8 or any miRNA or group of miRNAs set forth in Table 1, and analyzing comprises determining the levels of TSG101, INF-γ, IL1-β, TNF-a, IL-8 or any miRNA or group of miRNAs set forth in Table 1 in circulating EVs.

10. The method of claim 9, wherein analyzing comprises determining levels of hsa-miR-125 family, hsa-miR-335-3p, hsa-miR-339-5p, hsa-miR-4433b-5p, hsa-miR-130b-5p, hsa-miR-658, hsa-miR-6809, and/or hsa-miR-6510-5p in circulating EVs.

11. The method of claim 9, wherein the biomolecule is AAT and/or misfolded AAT aggregates, and analyzing comprises determining the level of total and polymeric AAT in circulating EVs.

12. The method of any of claims 1-11, wherein obtaining comprises obtaining EVs comprising ASGR1, and analyzing comprises determining the level of circulating EVs comprising ASGR1.

13. The method of any of claims 1-12, wherein obtaining comprises obtaining EVs comprising ASGR1; and further comprising determining the presence and/or level of one or more biomolecules in the circulating EVs.

14. The method of claim 13, wherein the one or more biomolecules comprise TSG101, INF-γ, IL1-β, TNF-a, IL-8, hsa-miR-125 family, hsa-miR-335-3p, hsa-miR-339-5p, hsa-miR-4433b-5p, hsa-miR-130b-5p, hsa-miR-658, hsa-miR-6809, and/or hsa-miR-6510-5p.

15. The method of any of claims 1-14, wherein the liver disease is liver fibrosis.

16. The method of any of claims 1-14, further comprising deriving a risk score for fibrosis by calculating an amount of differential presence of one or more biomarkers in the biological sample.

17. The method of claim 16, wherein the method comprises analyzing a biological sample from the subject using a predictive model based on the levels of one or more biomarkers.

18. The method of claim 17, wherein the predictive model is used to generate a Fibrosis Score and the Fibrosis Score is used to aid in the determination of the presence or absence of liver fibrosis in the subject.

19. A method of monitoring progression/regression of liver disease in a subject comprising: analyzing a first biological sample from a subject to determine the level(s) of one or more biomarkers for liver disease in the sample, and the first sample is obtained from the subject at a first time point; analyzing a second biological sample from a subject to determine the level(s) of the one or more biomarkers for liver disease, wherein the second sample is obtained from the subject at a second time point; and comparing the level(s) of one or more biomarkers in the first sample to the level(s) of the one or more biomarkers in the second sample in order to monitor the progression/regression of liver disease in the subject.

20. The method of claim 19, wherein the method further comprises comparing the level(s) of one or more biomarkers in the first sample, the level(s) of one or more biomarkers in the second sample, and/or the results of the comparison of the level(s) of the one or more biomarkers in the first and second samples to liver disease-positive and/or liver disease-negative reference levels of the one or more biomarkers.

21. A method of assessing the efficacy of a composition for treating liver disease comprising: analyzing, from a subject having liver disease and currently or previously being treated with a composition, a biological sample to determine the level(s) of one or more biomarkers for liver disease; and comparing the level(s) of the one or more biomarkers in the sample to (a) levels of the one or more biomarkers in a previously-taken biological sample from the subject, wherein the previously-taken biological sample was obtained from the subject before being treated with the composition, (b) liver disease-positive reference levels of the one or more biomarkers, and/or (c) liver disease-negative reference levels of the one or more biomarkers.

22. The method of claim 21, wherein the biological sample comprises EVs comprising ASGR1.

23. The method of claims 21 or 22, wherein the one or more biomarkers comprise at least one biomolecule comprising TSG101, INF-γ, IL1-β, TNF-a, IL-8, hsa-miR-125 family, hsa-miR-335-3p, hsa-miR-339-5p, hsa-miR-4433b-5p, hsa-miR-130b-5p, hsa-miR-658, hsa-miR-6809, and/or hsa-miR-6510-5p.

24. A method for assessing the efficacy of a composition in treating liver disease, comprising: analyzing a first biological sample from a subject to determine the level(s) of one or more biomarkers for liver disease, the first sample obtained from the subject at a first time point; administering the composition to the subject; analyzing a second biological sample from the subject to determine the level(s) of the one or more biomarkers, the second sample obtained from the subject at a second time point after administration of the composition; and comparing the level(s) of one or more biomarkers in the first sample to the level(s) of the one or more biomarkers in the second sample in order to assess the efficacy of the composition for treating liver disease.

25. A method for screening a composition for activity in modulating one or more biomarkers of liver disease, comprising: contacting one or more cells with a composition; analyzing at least a portion of the one or more cells or a biological sample associated with the cells to determine the level(s) of one or more biomarkers of liver; and comparing the level(s) of the one or more biomarkers with predetermined standard levels for the biomarkers to determine whether the composition modulated the level(s) of the one or more biomarkers.

26. The method of claim 25, wherein the predetermined standard levels for the biomarkers are level(s) of the one or more biomarkers in the one or more cells in the absence of the composition.

27. The method of claim 25, wherein the predetermined standard levels for the biomarkers are level(s) of the one or more biomarkers in one or more control cells not contacted with the composition.

28. The method of claim 25, wherein the method is conducted in vivo.

29. The method of claim 25, wherein the method is conducted in vitro.

30. A method according to any of claims 1-20, wherein the liver disease therapy comprises administering a composition that comprises one more agents selected from the group consisting of ACE inhibitors, alpha-tocopherol, interferon-alpha, PPAR-alpha agonists, anti-TGF-β1 monoclonal antibody (e.g. Fresolimumab), LOXL2 monoclonal antibody (e.g. AB0023 or GS-6624), IL-4/IL-13 dual antibody, LPA1 receptor antagonists, Av-beta-6 antibody, tyrosine kinase inhibitors, angiotensin receptor blockers, perferidone, obeticholic acid (OCALIVA), TIMP-1 antibody, PPAR-gamma agonists, TGf-beta inhibitors, Human pentraxin-2, Prolyl hydroxylase inhibitors, hedgehog inhibitors, CB1 inhibitors, Caspase inhibitors, or Galactin-3 inhibitor.

31. A method according to any of claims 21-29, wherein the composition comprises one or more agents selected from the group consisting of ACE inhibitors, alpha-tocopherol, interferon-alpha, PPAR-alpha agonists, anti-TGF-β1 monoclonal antibody (e.g. Fresolimumab), LOXL2 monoclonal antibody (e.g. AB0023 or GS-6624), IL-4/IL-13 dual antibody, LPA1 receptor antagonists, Av-beta-6 antibody, tyrosine kinase inhibitors, angiotensin receptor blockers, perferidone, obeticholic acid (OCALIVA), TIMP-1 antibody, PPAR-gamma agonists, TGf-beta inhibitors, Human pentraxin-2, Prolyl hydroxylase inhibitors, hedgehog inhibitors, CB1 inhibitors, Caspase inhibitors, or Galactin-3 inhibitor.

32. A method of obtaining EVs from blood or plasma that are produced by hepatocytes, the method comprising isolating or measuring a level of EVs in the blood or plasma that comprise ASGR1.

33. The method of claim 32, wherein the EVs comprise a higher level of ASGR1 compared to EVs produced from non-liver cells.

34. The method of claims 32 or 33, further comprising detecting one or more biomarkers in the EVs.

35. The method of claim 34, wherein the one or more biomarkers comprise detecting a presence or level of TSG101, INF-γ, IL1-β, TNF-a, IL-8, hsa-miR-125 family, hsa-miR-335-3p, hsa-miR-339-5p, hsa-miR-4433b-5p, hsa-miR-130b-5p, hsa-miR-658, hsa-miR-6809, and/or hsa-miR-6510-5p.

36. The method of any of claims 32-35, wherein the EVs are detected or measured using flow cytometry.

37. The method of any of claims 32-36, wherein when percentage of hepatocyte-derived EVs in the blood or plasma relative to total EVs in the blood or plasma is higher than 40 percent indicates liver disease.

38. The method of any of claims 32-36, wherein an amount of hepatocyte-derived EVs in the blood or plasma being higher compared to a control indicates liver disease.

39. The method of claim 38, wherein the control is a sample from a normal patient without liver disease.

40. A method comprising:
obtaining EVs from blood or plasma that are produced by hepatocytes, the method comprising isolating or measuring a level of EVs in the blood or plasma that comprise ASGR1 by subjecting the blood or plasma to flow cytometry, and
optionally detecting one or more biomarkers in the EVs; the one or more biomarkers comprise detecting a presence or level of TSG101, INF-γ, IL1-β, TNF-a, IL-8, hsa-miR-125 family, hsa-miR-335-3p, hsa-miR-339-5p, hsa-miR-4433b-5p, hsa-miR-130b-5p, hsa-miR-658, hsa-miR-6809, and/or hsa-miR-6510-5p.
